(19) United States
(12) Patent Application Publication
(10) Pub. No.: US 2023/0149614 A1
(43) Pub. Date: May 18, 2023

(54) METHOD OF REMOVING HARMFUL SUBSTANCES IN BLOOD

(71) Applicant: UNIST(ULSAN NATIONAL INSTITUTE OF SCIENCE AND TECHNOLOGY), Ulsan (KR)

(72) Inventors: Joo Hun Kang, Ulsan (KR); Se Yong Kwon, Ulsan (KR); Bong Hwan Jang, Ulsan (KR); Min Seok Lee, Ulsan (KR)

(73) Assignee: UNIST(ULSAN NATIONAL INSTITUTE OF SCIENCE AND TECHNOLOGY), Ulsan (KR)

(21) Appl. No.: 18/156,965

(22) Filed: Jan. 19, 2023

Related U.S. Application Data
(63) Continuation of application No. PCT/KR2020/009519, filed on Jul. 20, 2020.

(51) Int. Cl. A61M 1/34

(52) U.S. Cl. CPC A61M 1/3406

(57) ABSTRACT

A method of removing harmful substances in blood according to an embodiment of the present disclosure is performed by a fluidic device including an inlet into which isolated blood is injected and at least one blood-clot generating and fixing unit, and the method includes injecting isolated blood into the inlet and removing harmful substances in blood by generating blood clots in the at least one blood-clot generating and fixing unit from the injected blood and fixing the blood clots.

### CROSS REFERENCE TO RELATED APPLICATION

This application is a continuation of International Patent Application No. PCT/KR2020/009519, filed Jul. 20, 2020. The prior application is incorporated herein by reference in its entirety.

### FIELD

The present disclosure relates to a method of removing harmful substances in blood.

### BACKGROUND

Infectious diseases refer to disorders caused by inflow of pathogens, such as bacteria, fungi, viruses, parasites, and other pathogenic substances, outside the body into the human body. In the treatment of sepsis among infectious diseases, the survival rate per hour decreases by 7.6% whenever the use of the correct treatment material is delayed. In the case of a culture method which is a method of identifying sepsis-causing pathogens, it takes at least 24 hours or more than 48 hours to culture bacteria, meaning that a large amount of time is required, and even when culture by the method is completed, pathogens are not detected in some cases, and thus false negative results commonly occur. To overcome these limitations, after the presence of sepsis is determined, treatment is performed by administering a large amount of various types of antibiotics. However, a treatment prognosis is not good, and side effects occur due to massive doses. In addition, diseases such as MERS and SARS have a high mortality rate, but treatment drugs have not been developed or it hasn't been long since treatment drugs have been developed, and many side effects have been reported.

Therefore, it is necessary to develop a new treatment method that can significantly lower fatality rates of urgent infectious diseases and involves little or no side effects.

### SUMMARY

There is provided a method that can be applied to a new treatment method that has no or minimized side effects by removing harmful substances in blood by using the human body's immune mechanism that appears in a process of blood clotting.

A method of removing harmful substances or blood clots in blood according to an embodiment of the present disclosure includes injecting isolated blood, and removing harmful substances in blood by generating blood clots from the injected blood, and fixed blood clots capture the harmful substances in the blood being continuously injected, or removing blood clots in blood by fixing the blood clots existing in the blood from the injected blood.

The subject matter of this disclosure can be used in connection with various diseases including sepsis, viremia, and infectious diseases by not only removing blood blots from blood without injecting other substances, such as drugs, into the human body, but also removing harmful substances by using the human body's immune mechanism.

The subject matter of this disclosure can be used to form blood clots by generating shear stress in blood. Accordingly, since harmful substances in blood can be removed by using the human body's immune mechanism that appears in a process of blood clotting, the disclosure can be applied to various treatment methods, such as hemodialysis, or medical devices.

### BRIEF DESCRIPTION OF THE DRAWINGS

FIG. 1 is a schematic view of a fluidic device according to an embodiment of the present disclosure.

FIG. 2 is a plan view of a fluidic device according to an embodiment of the present disclosure.

FIG. 3 is a diagram of a fluidic device according to an embodiment of the present disclosure.

FIG. 4 is a diagram of a fluidic device according to an embodiment of the present disclosure.

FIG. 5 is a diagram of a fluidic device according to an embodiment of the present disclosure.

FIG. 6 is a diagram of a fluidic device capable of generating blood clots by inducing shear stress, according to an embodiment of the present disclosure.

FIG. 7 is a diagram of a fluidic device including a plurality of layers, according to an embodiment of the present disclosure.

FIG. 8 is a diagram of a fluidic device including particles in a blood-clot generating and fixing unit, according to an embodiment of the present disclosure.

FIG. 9a schematically shows a bacteria removal experiment using the fluidic device of FIG. 8.

FIGS. 9b and FIG. 9c show the results of the experiment according to FIG. 9a.

FIG. 10 is a diagram of a fluidic device capable of generating blood clots by inducing shear stress, according to an embodiment of the present disclosure.

FIG. 11 is a diagram of a fluidic device including a blood-clot generating and fixing unit having a varying cross-sectional area, according to an embodiment of the present disclosure.

FIG. 12 is a diagram of a fluidic device including a plurality of layers, according to an embodiment of the present disclosure.

FIG. 13 is a diagram of a fluidic device in the form of a tube bundle, according to an embodiment of the present disclosure.

FIG. 14 is a diagram showing a process in which blood clots are generated in a fluidic device according to an embodiment of the present disclosure.

FIG. 15 is a diagram showing a process of removing harmful substances in blood by a fluidic device according to an embodiment of the present disclosure.

FIG. 16 is a schematic diagram of a preliminary in-vitro blood circulation experiment using a fluidic device according to an embodiment of the present disclosure.

FIG. 17 is a graph showing a removal rate of harmful substances in blood using a fluidic device according to an embodiment of the present disclosure.

FIG. 18 is a diagram showing a simulation result of shear stress at an inlet of a fluidic device according to an embodiment of the present disclosure.

FIG. 19 is a diagram showing a surface shear rate or flow streamline of a fluid according to each microstructure, as a result of fluid simulation of a fluidic device according to an embodiment of the present disclosure.

FIG. 20 shows pictures of a blood experiment result of a fluidic device an embodiment of the present disclosure and blood-clots generated in the fluidic device.

FIG. 21 is a diagram of a microstructure of a blood-clot generating and fixing unit of a fluidic device according to an embodiment of the present disclosure, and arrows indicate a direction in which blood flows.

FIG. 22 shows graphs showing efficiency of removing various types of bacteria by a fluidic device according to an embodiment of the present disclosure through decreases in relative quantity of the bacteria.

FIG. 23 is a graph showing Staphylococcus aureus removal efficiency of a fluidic device according to an embodiment of the present disclosure through a decrease in relative quantity of bacteria.

FIG. 24 is a graph of measuring hemolysis of rat blood by a fluidic device according to an embodiment of the present disclosure.

### DETAILED DESCRIPTION

The term “blood” as used herein may include whole blood, artificial blood, pretreated blood (for example, blood pretreated with an anticoagulant), some components of blood, such as plasma, plasma proteins, blood corpuscles, or blood cells, or lymph fluid, cerebrospinal fluid, or bone marrow, which may include blood or some components of blood.

The term “isolated blood” as used herein may refer to blood that is isolated from the inside of an entity to the outside of the body, or blood that is isolated to the outside of the body and circulates.

The term “apparatus or method for removing harmful substances” as used herein may include an apparatus or method for removing harmful substances from blood, and a method or apparatus for detecting harmful substances after removing the harmful substances.

A method of removing harmful substances or blood clots in blood according to an embodiment of the present disclosure is performed by a fluidic device including an inlet into which isolated blood is injected and at least one blood-clot generating and fixing unit, and the method includes injecting isolated blood into the inlet, and removing harmful substances in blood by generating blood clots in the at least one blood-clot generating and fixing unit from the injected blood and fixing the blood clots, wherein, in the removing of harmful substances in blood, the generated blood clots and the fixed blood clots capture the harmful substances in the blood being continuously injected, and the removing of harmful substances in blood includes allowing the blood to pass through the at least one blood-clot generating and fixing unit, injecting substances that generate blood clots into the blood, or contacting a surface on which the blood clots are generated with the injected blood.

In an embodiment, the fluidic device may include a fluidic channel through which blood flows from the inlet and/or a fluidic channel arranged to correspond to the at least one blood-clot generating and fixing unit and through which blood moves.

In an embodiment, the generated blood clots and the fixed blood clots may remove the blood clots in the blood by fixing the blood clots in the blood being continuously injected.

In an embodiment, the substances that generate blood clots and/or the surface on which the blood clots are generated may include at least one selected from the group consisting of glass, polymers, metals, calcium, von Willebrand factor, blood cells, plasma, platelets, blood clotting factors, prothrombin, collagen, thrombin, fibronectin, fibrinogen, fibrin, neutrophil extracellular traps (NETs), antibiotics, heparin, heparan sulfate, chitosan, sialic acid, hyaluronic acid, polyethylene imine (PEI), dextran sulfate, chondroitin sulfate, dermatan sulfate, glycosaminoglycan, mannose, montmorillonite, bentonite, nanoclay, ligands including urea bonds, thiourea bonds, amide bonds, peptide bonds, amino groups, amide groups, carboxyl groups, pyridyl groups, pyrimidyl groups, and imidazole groups, polymyxin B, and polyamino compounds.

The metals may include metal particles, and the type of the metals may be selected from the group consisting of gold (Au), silver (Ag), copper (Cu), platinum (Pt), palladium (Pd), nickel (Ni), cobalt (Co), iridium (Ir), osmium (Os), ruthenium (Ru), iron (Fe), rhodium (Rh), and alloys thereof. In addition, the polymers may include cross-linked polymers or polymer particles, and examples of the polymers may include natural polymers or synthetic polymers. Specifically, examples of the natural polymers may include starch, cellulose, protein, DNA, or natural rubber. In addition, examples of the synthetic polymers are silicone organic polymers, for example, a linear or cyclic silicone having 2 to 7 silicon atoms, wherein the silicone may be a silicone organic polymer optionally including an alkyl or alkoxy group having 1 to 10 carbon atoms. Monomers of the synthetic polymers may be selected from ethylene glycol, diethylene glycol, ethylene, styrene, vinyl chloride, alkyl (meth)acrylate, (meth)acrylamide, vinyl acetate, alkenyl (meth)acrylate, aryl (meth)acrylate, alkylaryl (meth)acrylate, amine-containing (meth)acrylate, phosphorus-containing (meth)acrylate, sulfur-containing (meth)acrylate, vinyl aromatic, (meth)acrylic acid, substituted ethylene, vinyl imidazole, norbornene, substituted norbornene, olefins, pentaerythritol tetraacrylate, trimethylolpropane triacrylate (TMPTA), trimethylolpropane trimethacrylate (TMPTMA), diethylene glycol diacrylate, diethylene glycol dimethacrylate, triethylene glycol diacrylate, triethylene glycol dimethacrylate, siloxanes, dimethylsiloxanes, and combinations thereof. In addition, the glass may include glass particles. The glass particles, the polymer particles, or the metal particles may be spherical or amorphous and may have a size of about 1 nm to about 5 cm, about 100 μm to about 1 cm, about 1 nm to about 1 cm, about 1 μm to about 5 cm, about 1 μm to about 1 cm, about 10 nm to about 100 μm, or about 100 nm to about 100 μm.

Bringing injected blood into contact with substances capable of generating blood clots may be to generate blood clots by a chemical mechanism, and bringing injected blood into contact with a structure configured to generate blood clots may be to generate blood clots by a physical mechanism. The generation of the blood clots by the chemical mechanism and the physical mechanism may be performed together. For example, the substances capable of generating blood clots may be surface-treated (coated) on at least a portion or surface of the at least one blood-clot generating and fixing unit or at least a portion of a substrate of the fluidic device. In addition, for example, the at least one blood-clot generating and fixing unit may include a microstructure configured to immobilize blood clots that are generated due to a contact between the substances capable of generating blood clots and blood. Therefore, a plurality of microstructures may be configured to fix generated blood clots. The structure or microstructure configured to generate blood clots with the injected blood will be described in detail below.

In an embodiment, substances capable of generating blood clots or binding to blood clots may adhere to or surface-treated on at least a portion or surface of the at least one blood-clot generating and fixing unit, such that generated blood clots may be fixed to the at least one blood-clot generating and fixing unit.

In an embodiment, the at least one blood-clot generating and fixing unit may generate blood clots depending on shear stress of blood according to a change in flow rate of the blood in which the flow rate of the blood increases or decreases as the blood passes through the at least one blood-clot generating and fixing unit. In other words, the fixing of the blood clots existing in the blood from the injected blood may include increasing or inducing shear stress of the injected blood or bringing the at least one blood-clot generating and fixing unit configured to be able to fix blood clots into contact with the injected blood.

As the flowing blood flows from a tube (for example, the inlet) having a large cross-sectional area into the fluidic device, a flow rate increases due to a difference in cross-sectional area. As a result, due to increased shear stress, substances involved in blood clotting are activated and generate blood clots, or while passing through the at least one blood-clot generating and fixing unit, the generated blood clots may adhere to the surface thereof or may be captured by gaps between the microstructures of the at least one blood-clot generating and fixing units, such that the blood clots may be fixed. In addition, since other blood clots may adhere to surfaces of the fixed blood clots, blood clots may be gradually fixed more widely around the fixed blood clots. In addition, as the injected blood passes through the plurality of microstructures between minute gaps, a shear rate increases, such that generation of blood clots may be promoted. Therefore, blood clots generated near the inlet may be trapped and fixed between the microstructures, or blood clots may be generated due to high shear stress occurring between the microstructures. Therefore, both mechanisms may serve to generate and fix blood clots within the fluidic device.

In addition, the microstructure of the at least one blood-clot generating and fixing unit causes a change in flow rate of the injected blood, and the greater the change, the higher the surface shear rate of the blood, resulting in generation of blood clots. A surface shear rate of the blood shows the largest value between the plurality of microstructures, and the blood clots may be fixed to a surface or periphery of the microstructure.

In an embodiment, shear stress of blood, which may generate blood clots from the flowing blood, may be about 1 dyne/cm² to about 10,000 dyne/cm², about 1 dyne/cm² to about 5,000 dyne/cm², about 1 dyne/cm² to about 2,000 dyne/cm², about 2 dyne/cm² to about 1,500 dyne/cm², about 10 dyne/cm² to about 1,500 dyne/cm², about 50 dyne/cm² to about 1,000 dyne/cm², about 100 dyne/cm² to about 1,000 dyne/cm², or about 200 dyne/cm² to about 1000 dyne/cm².

In an embodiment, a flow rate of the injected blood may be any flow rate as long as it is sufficient to generate blood clots, and may be, for example, about 0.1 μm/sec to about 2,600,000 μm/sec, about 0.1 μm/sec to about 1,500,000 μm/sec, about 0.1 μm/sec to about 1,000,000 μm/sec, about 0.1 μm/sec to about 500,000 μm/sec, about 0.1 μm/sec to about 100,000 μm/sec, about 0.1 μm/sec to about 60,000 μm/sec, about 1 μm/sec to about 60,000 μm/sec, about 10 μm/sec to about 40,000 μm/sec, about 100 μm/sec to about 20,000 μm/sec, about 200 μm/sec to about 20,000 μm/sec, or about 400 μm/sec to about 10,000 μm/sec.

The term “blood clots” as used herein refers to a product produced through a blood clotting process outside or inside the body, and may include an embolus. The blood clots may include artificially generated blood clots and blood clots originally existing in the blood. The blood clots may include at least one selected from the group consisting of blood cells, plasma, platelets, calcium, von Willebrand factor, blood clotting factors, prothrombin, collagen, thrombin, fibronectin, fibrinogen, fibrin, neutrophil extracellular traps (NETs), antibiotics, heparin, heparan sulfate, chitosan, sialic acid, hyaluronic acid, polyethylene imine (PEI), dextran sulfate, chondroitin sulfate, dermatan sulfate, glycosaminoglycan, mannose, montmorillonite, bentonite, nanoclay, ligands including urea bonds, thiourea bonds, amide bonds, peptide bonds, amino groups, amide groups, carboxyl groups, pyridyl groups, pyrimidyl groups, and imidazole groups, polymyxin B, and polyamino compounds. One of components included in the blood clots may capture (remove or bind to) harmful substances in flowing blood.

The term “harmful substances” as used herein may include at least one selected from the group consisting of infectious substances, cancer, tumors, cancer cells, cancer-causing substances, cancer-related factors, and extracellular vesicles (exosome). The infectious substances may include substances estimated to contain pathogens, pathogen-containing substances, pathogens, and non-pathogens. Examples of the harmful substances may include microorganisms or prions, and more specifically, may include bacteria, parasites, viruses, fungi, rickettsiae, and algae. The above-described substances exist on surfaces of blood clots or within blood clots, and these may non-specifically bind to harmful substances or capture harmful substances. Therefore, there is an effect of removing harmful substances or blood clots in a non-specific way using the human body's immune mechanism without introduction of drugs or additional substances into the human body.

The term “microstructure” as used herein may refer to a blood-flow related structure formed in a microfluid. The microstructure may protrude from an upper surface of a substrate.

A cross section of the microstructure may be n-gonal or amorphous, and n may be 3 to 12. The plurality of microstructures may have the same cross section, height, and interval, or may have different cross sections, heights, and intervals. The plurality of microstructures may have any form as long as they may generate blood clots from flowing blood by increasing a surface shear rate of the flowing blood and fix the generated blood clots.

In a detailed embodiment, a cross section of the microstructure may include, for example, a triangle, a rhombus, a quadrangle, a pentagon, a hexagon, a heptagon, an octagon, an alphabet H shape, or a bow-shaped octagon having at least two sides placed inward in the cross section.

In an embodiment of the present disclosure, a height or interval of the microstructure may be about 0.1 μm to about 10,000 μm, about 0.1 μm to about 400 μm, about 0.1 μm to about 200 μm, about 0.1 μm to about 180 μm, about 0.2 μm to about 180 μm, about 0.5 μm to about 150 μm, about 1 μm to about 150 μm, or about 10 μm to about 150 μm. A height of the structure may be equal to a height of the fluidic device. An interval between the plurality of microstructures may refer to a distance between a point of one microstructure and the same point of another microstructure. The interval between the microstructures may be sufficient for one cell or two cells to pass. A length of one side of a cross section of the microstructure may be at least about 0.1 μm, for example, about 0.1 μm to about 10,000 μm, about 0.1 μm to about 1,000 μm, about 0.1 μm to about 800 μm, about 0.1 μm to about 600 μm, or about 0.1 μm to about 400 μm.

In another detailed example, the microstructure may be one in which microstructures having the same shape are uniformly formed (at the same or similar intervals or heights) in the fluidic device. In addition, the fluidic device may include a fluidic channel arranged to correspond to the at least one blood-clot generating and fixing unit and through which blood moves, and a height of the microstructure may be the same as a height of the fluidic channel.

In an embodiment, the particles may be rod-shape, bead-shaped, or fibers. The particles may be used without limitation as long as they are configured to form a gap in the at least one blood-clot generating and fixing unit and may induce shear stress of blood. Examples of the particles may include plastic rods, plastic beads, metal rods, metal beads, glass rods, glass beads, or glass fibers. Without being limited to a particular theory, shear stress of blood flowing through a gap between the beads occurs, such that blood clots may be generated, and harmful substances in the blood may be removed by the generated blood clots.

In another detailed example, a cross-sectional area of a fluidic channel of the at least one blood-clot generating and fixing unit may be smaller than a cross-sectional area of a fluidic channel of the inlet, or a height of a fluidic channel of the at least one blood-clot generating and fixing unit may be smaller than a diameter of a cross section of a fluidic channel of the inlet. As described above, as flowing blood flows from a tube (for example, the inlet) having a large cross-sectional area into a microfluidic chip, a flow rate and shear stress increase due to a difference in cross-sectional area, such that substances involved in blood clotting are activated and may generate blood clots. Therefore, as blood flows from the inlet to the at least one blood-clot generating and fixing unit, shear stress of the blood is induced, such that blood clots may be generated at a part where the inlet and the at least one blood-clot generating and fixing unit are connected to each other.

In another detailed example, the at least one blood-clot generating and fixing unit may include a structure in which a cross-sectional area of a fluidic channel is changed at least once. In addition, the at least one blood-clot generating and fixing unit may include a plurality of layers or a plurality of tubes. For example, the at least one blood-clot generating and fixing unit may include at least two layers or tubes, at least five layers or tubes, at least eight layers or tubes, at least 10 layers or tubes, at least 15 layers or tubes, at least 20 layers or tubes, or at least 30 layers or tubes.

In another detailed example, a blood clot filter capable of fixing blood clots to the at least one blood-clot generating and fixing unit or an outlet may be further included. When the at least one blood-clot generating and fixing unit does not include a separate structure for fixing blood clots or surface treatment for fixing blood clots, the blood clot filter capable of fixing blood clots to the at least one blood-clot generating and fixing unit or an outlet may be arranged to prevent generated blood clots or blood clots originally existing in blood from passing and leaking out of the filter and immobilize the blood clots in the filter, such that the blood clots or harmful substances in the blood may be removed. The blood clot filter may have a porosity of about 1% to about 99%, a particle holding size of about 10 μm to about 20,000 μm, and a diameter of a through hole of about 10 μm to about 20,000 μm.

The blood clot filter may remove (or fix) particles (for example, blood clots) having a size of at least about 10 μm, at least about 50 μm, at least about 100 μm, at least about 500 μm, at least about 1,000 μm, at least about 2,000 μm, at least about 5,000 μm, at least about 8,000 μm, or at least about 10,000 μm.

The fluidic device may be manufactured from polydimethylsiloxane (PDMS), polyethersulfone (PES), poly(3,4-ethylenedioxythiophene), poly(styrenesulfonate), polyimide, polyurethane, polyester, perfluoropolyether (PFPE), polycarbonate, or combinations of the polymers.

A structure in the microfluid may be manufactured by lithography (for example, photolithography or soft lithography), hot embossing, extrusion, or the like.

Various modifications may be made to embodiments, and specific embodiments are illustrated in the drawings and will be described in detail in the detailed description. The effect and features of the embodiments, and a method to achieve the same, will be clearer referring to the detailed descriptions below with the drawings. However, the present embodiments may be implemented in various forms, not by being limited to the embodiments presented below.

Hereinafter, embodiments will be described in detail with reference to the accompanying drawings, and in the description with reference to the drawings, the same or corresponding components are indicated by the same reference numerals and redundant descriptions thereof are omitted.

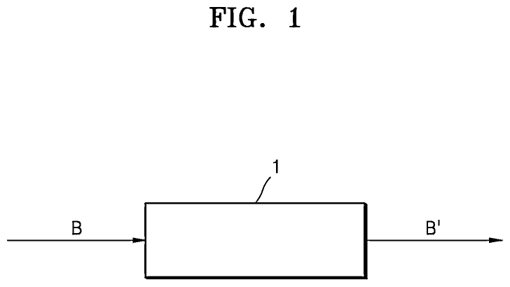

FIG. 1 is a schematic view of a fluidic device 1 according to an embodiment of the present disclosure.

Referring to FIG. 1, when blood B passes through the fluidic device 1, blood clots are generated inside the fluidic device 1, and the generated blood clots may be fixed inside the fluidic device 1. As the blood B passes through the fluidic device 1, shear stress occurs, and blood clots may be generated by the shear stress.

The fluidic device 1 includes an inlet into which the blood B flows and an outlet through which the blood B is discharged. In addition, the fluidic device 1 may include a guide connecting the inlet and the outlet together and defining a fluidic channel through which the blood B flows, and a blood-clot generating and fixing unit provided in plurality in a length direction of the guide and configured to induce substances that may generate blood clots or shear stress that may generate blood clots by contacting with the blood B flowing inside the fluidic channel or configured to fix blood clots. The blood-clot generating and fixing unit generates and fixes blood clots by changing shear stress by contacting with the blood B, and harmful substances existing in the blood B may be captured in the generated blood clots.

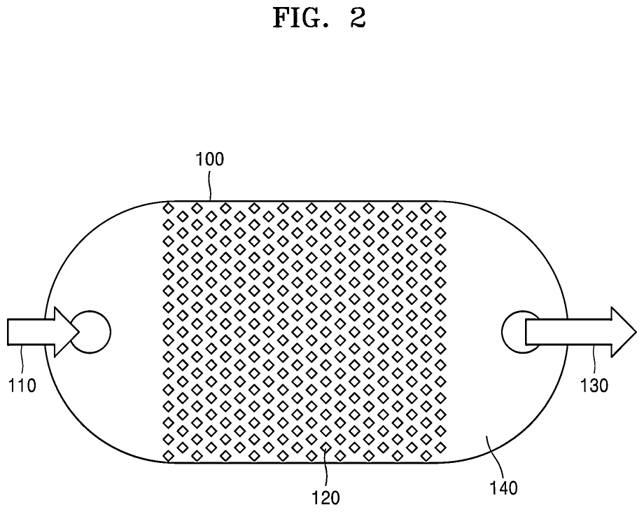

FIG. 2 is a plan view of a fluidic device 100 according to an embodiment of the present disclosure.

Referring to FIG. 2, there is provided the fluidic device 100 for removing harmful substances in blood, wherein the fluidic device 100 according to an embodiment of the present disclosure includes an inlet 110 into which blood isolated from the body is injected, a blood-clot generating and fixing unit 140 including a structure configured to induce substances that may generate blood clots or shear stress that may generate blood clots by contacting with the blood or configured to fix blood clots, and an outlet 130 to discharge the blood from the blood-clot generating and fixing unit 140. The inlet 110 may include a first channel as a fluidic channel, and the outlet 130 may include a second channel as a fluidic channel. Each of the first channel and the second channel may be connected to the blood-clot generating and fixing unit 140.

In addition, the blood-clot generating and fixing unit 140 may include a microstructure 120 protruding from an upper surface of the fixing unit 140 to induce shear stress or to fix blood clots, as the structure configured to induce shear stress that may generate blood clots or configured to fix blood clots.

In addition, the blood-clot generating and fixing unit 140 may include substances that may generate blood clots or may bind to blood clots. The substances that may generate blood clots or may bind to blood clots may be surface-treated (coated) on a substrate of the blood-clot generating and fixing unit 140, or on at least a portion within a channel thereof, or on the structure configured to induce shear stress that may generate blood clots, or may be coated on a plurality of microstructures 120 or at least a portion of particles.

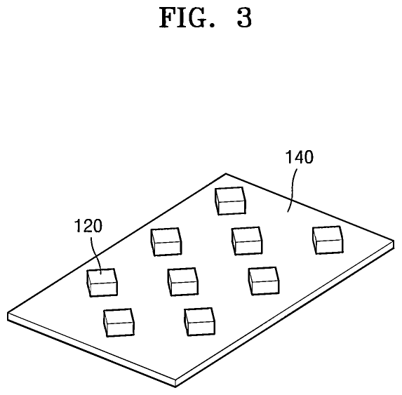

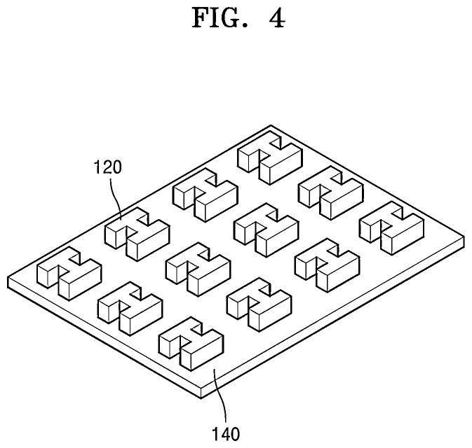

Referring to FIGS. 3 to 5, cross sections of the plurality of microstructures 120 may be n-gonal or amorphous, and n may be 3 to 12. The plurality of microstructures 120 may have the same cross section, height, and interval, or may have different cross sections, heights, and intervals. The plurality of microstructures 120 may have any form as long as they may generate blood clots from flowing blood by changing a surface shear rate of the flowing blood and may fix the generated blood clots. In an embodiment, a cross section of the microstructure 120 may include, for example, a triangle, a rhombus, a quadrangle, a pentagon, a hexagon, a heptagon, an octagon, an alphabet H shape, or a bow-shaped octagon having at least two sides placed inward in the cross section. In addition, a height of the microstructure 120 may be the same as a height of the channel.

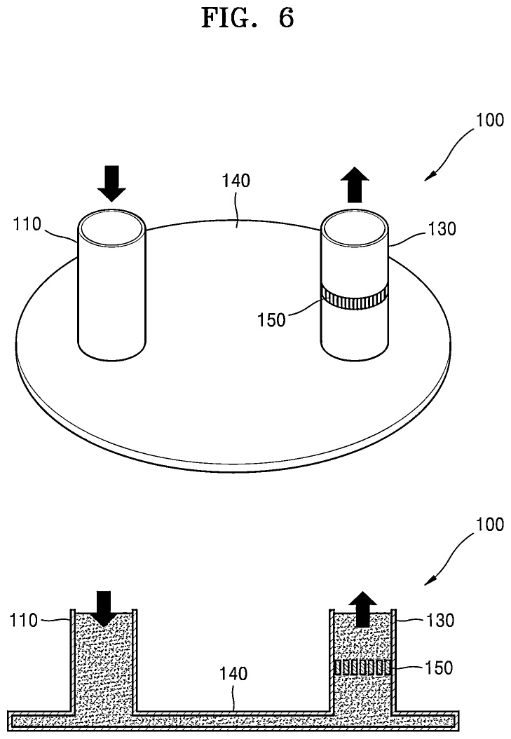

Referring to FIG. 6, as a structure configured to induce shear stress that may generate blood clots, a cross section of a fluidic channel of the blood-clot generating and fixing unit 140 may be smaller than a cross section of a fluidic channel of the inlet 110. As described above, as flowing blood flows from a tube (for example, the inlet) having a large cross-sectional area into the fluidic device 100, a flow rate and shear stress increase due to a difference in cross-sectional area. Thus, substances involved in blood clotting are activated and may generate blood clots. Therefore, as blood flows from the inlet 110 to the blood-clot generating and fixing unit 140, shear stress of the blood is changed, and accordingly, blood clots may be generated at a part where the inlet 110 and the blood-clot generating and fixing unit 140 are connected to each other. The fluidic device 100 may further include a blood clot filter 150 that may fix blood clots to the blood-clot generating and fixing unit 140 or the outlet 130. When the blood-clot generating and fixing unit 140 does not include a separate structure or surface treatment for fixing blood clots, the blood clot filter 150 that may fix blood clots to the blood-clot generating and fixing unit 140 or the outlet 130 may be arranged to prevent the generated blood clots or blood clots originally existing in blood from passing and immobilize the blood clots, such that the blood clots or harmful substances in the blood may be removed, or even when the blood-clot generating and fixing unit 140 includes a separate structure or surface treatment for fixing blood clots, the blood clot filter 150 may be arranged to more effectively immobilize residual blood clots, such that blood clots or harmful substances in blood may be removed.

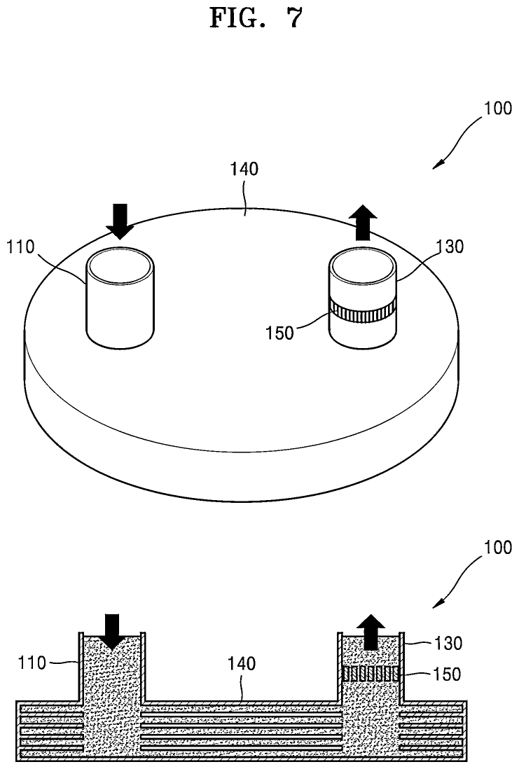

In addition, referring to FIG. 7, the blood-clot generating and fixing unit 140 may include a plurality of layers.

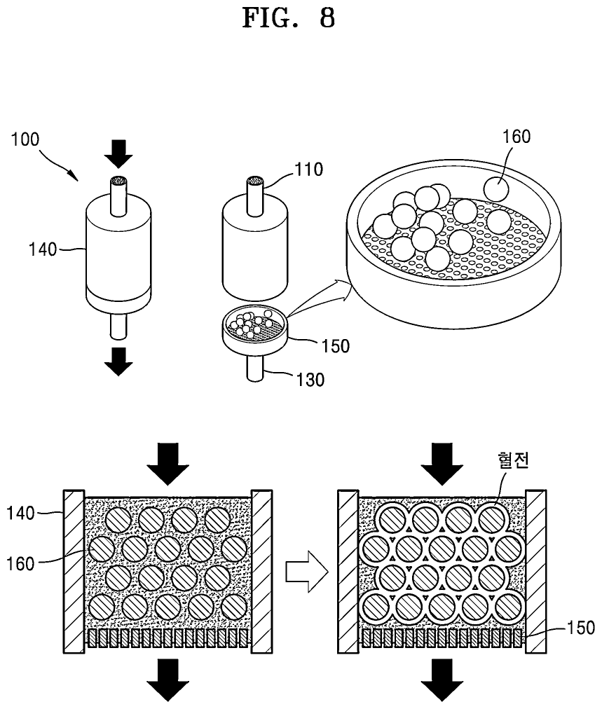

In addition, referring to FIG. 8, the blood-clot generating and fixing unit 140 may include particles 160 that are not immobilized in the blood-clot generating and fixing unit 140 and may induce shear stress of blood, as a material or structure configured to induce shear stress that may generate blood clots or configured to fix blood clots. The particles 160 may be rod-shaped, bead-shaped, or fibers. The particles 160 may be used without limitation as long as the particles 160 are configured to form a gap in the blood-clot generating and fixing unit 140, and may induce shear stress of blood or may adhere to blood clots. Examples of the particles 160 may include polymer rods, polymer beads, glass rods, glass beads, metal rods, metal beads, or glass fibers. Without being limited to a particular theory, shear stress of blood flowing through gaps between the particles 160 occurs, resulting in generation of blood clots, and harmful substances or blood clots in the blood may be removed by the generated blood clots, or blood clots or substances that may generate blood clots may be attached to surfaces of the particles 160 or the particles 160 themselves may adhere to blood clots, such that harmful substances or blood clots in blood may be removed.

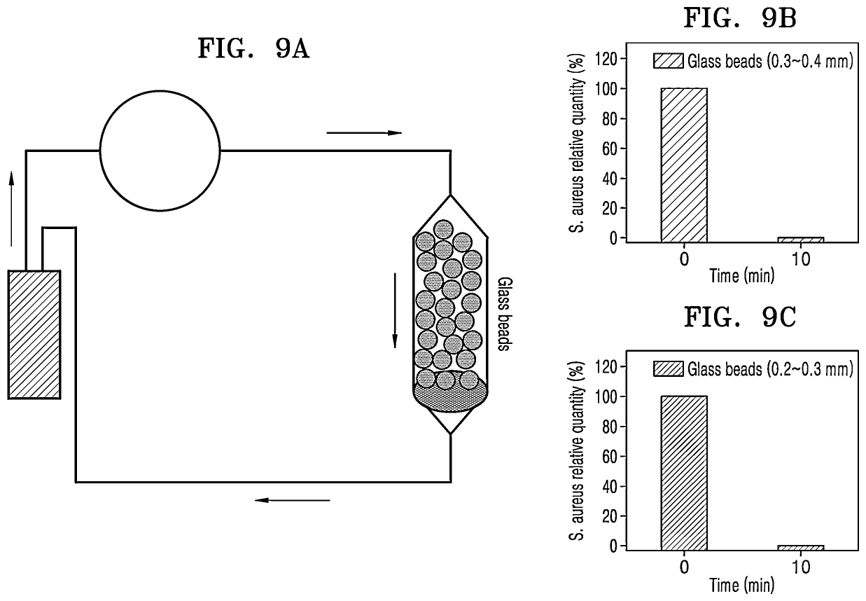

FIG. 9 is a diagram showing results of bacteria removal experiments using the fluidic device 100 of FIG. 8.

As shown in FIGS. 8 and 9, bacteria removal experiments were conducted using the fluidic device 100 including glass beads as the particles 160. More specifically, as shown in (a) of FIG. 9, the fluidic device 100 was fully filled with the particles 160 which are glass beads, and blood was flowed into the fluidic device 100 using a pump. The fluidic device 100 had a filter having a hole size of 70 μm in one cross section of a silicone tube having an inner diameter of 4.8 mm and a length of 10 cm. The filter allowed the particles 160 to stay without leaking out of the fluidic device 100.

To prepare a human blood sample as an infection model, 1×10⁴-2×10⁴ CFU/ml of Staphylococcus aureus was mixed with 10 ml of human blood (from Ulsan Blood Center of Korean Red Cross), and then a 5 mM CaCl₂ solution was added thereto. A silicone tube having an inner diameter of 1.6 mm was connected to an inlet and outlet of the fluidic device 100, and bacteria-infected blood (shaken at a temperature of 4° C. in a Thermomixer C (Eppendorf, USA)) was circulated (20 ml/h) using a peristaltic pump (Reglo Digital). The bacteria were mixed with the blood, a sample of the blood was collected before circulation in the device and after 10 minutes of circulation, and a concentration of the bacteria in the blood was measured by agar plating.

(b) of FIG. 9 is a graph of Staphylococcus aureus removal efficiency after 10 minutes of the blood circulation when the particles 160 having a size of 0.3-0.4 mm were used. (c) of FIG. 9 is a graph of Staphylococcus aureus removal efficiency after 10 minutes of the blood circulation when the particles 160 having a size of 0.2-0.3 mm were used. As shown in (b) and (c) of FIG. 9, it was found that Staphylococcus aureus was reduced by almost 100% after 10 minutes, compared to the initial concentration.

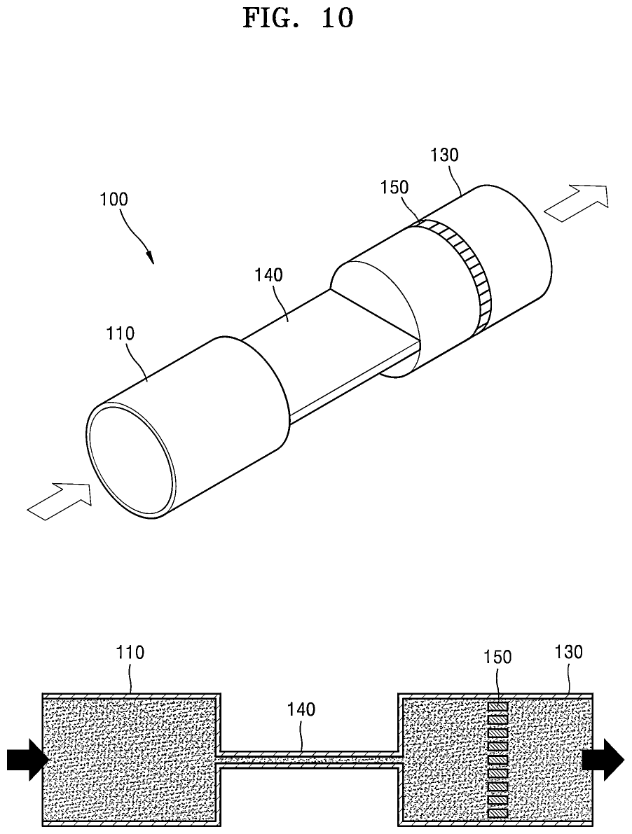

Referring to FIG. 10, as a structure configured to induce shear stress that may generate blood clots, a cross section of a fluidic channel of the blood-clot generating and fixing unit 140 may be smaller than a cross section of a fluidic channel of the inlet 110. The principle of blood clot generation by the above-described structure has been described with reference to FIG. 6.

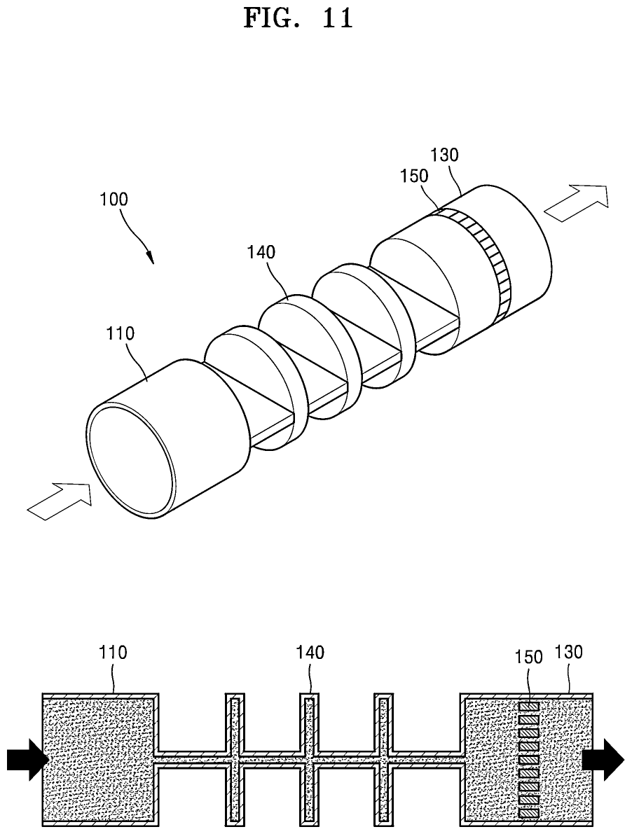

Referring to FIG. 11, the blood-clot generating and fixing unit 140 may include a structure in which a cross-sectional area of the fluidic channel is changed at least once. Through this change in cross-sectional area, change in shear stress may be made more diverse.

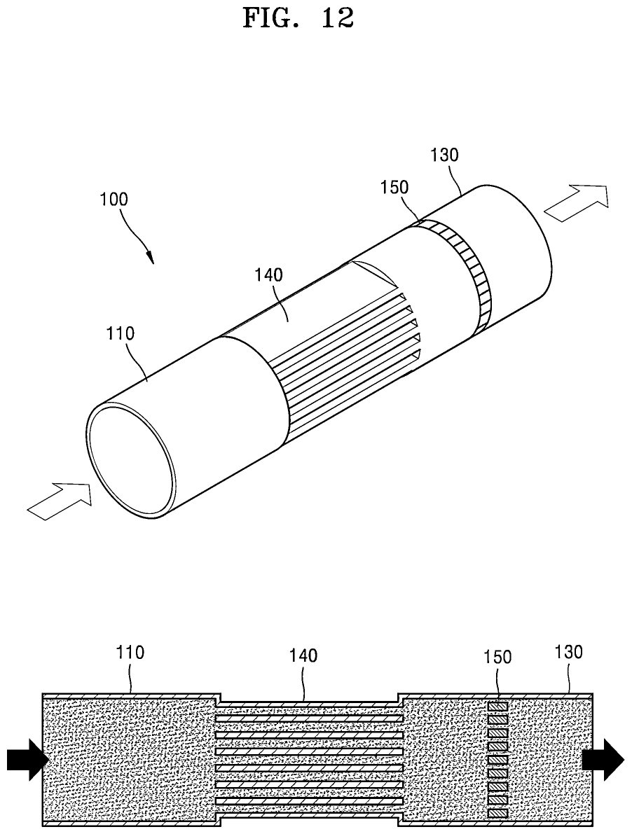

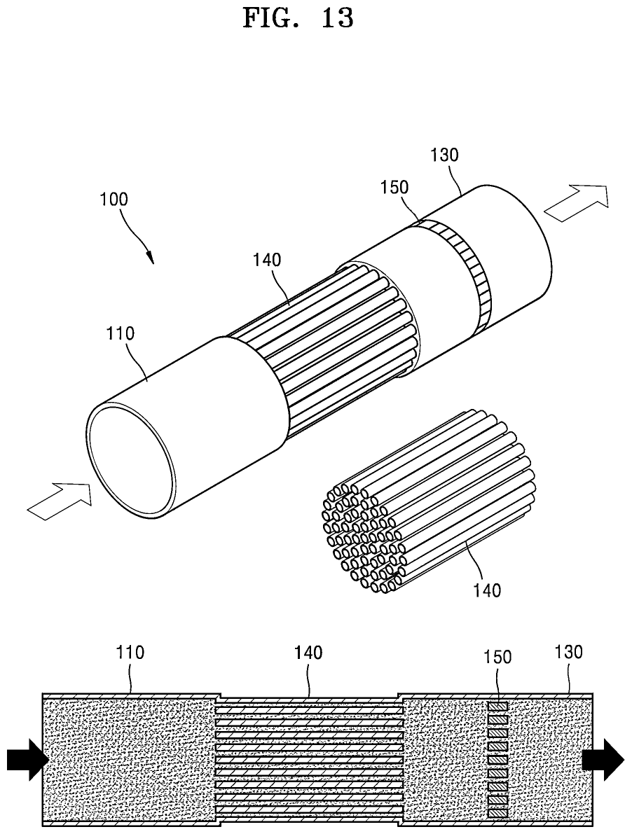

Referring to FIGS. 12 and 13, the blood-clot generating and fixing unit 140 may include a plurality of layers or a plurality of tubes (channels).

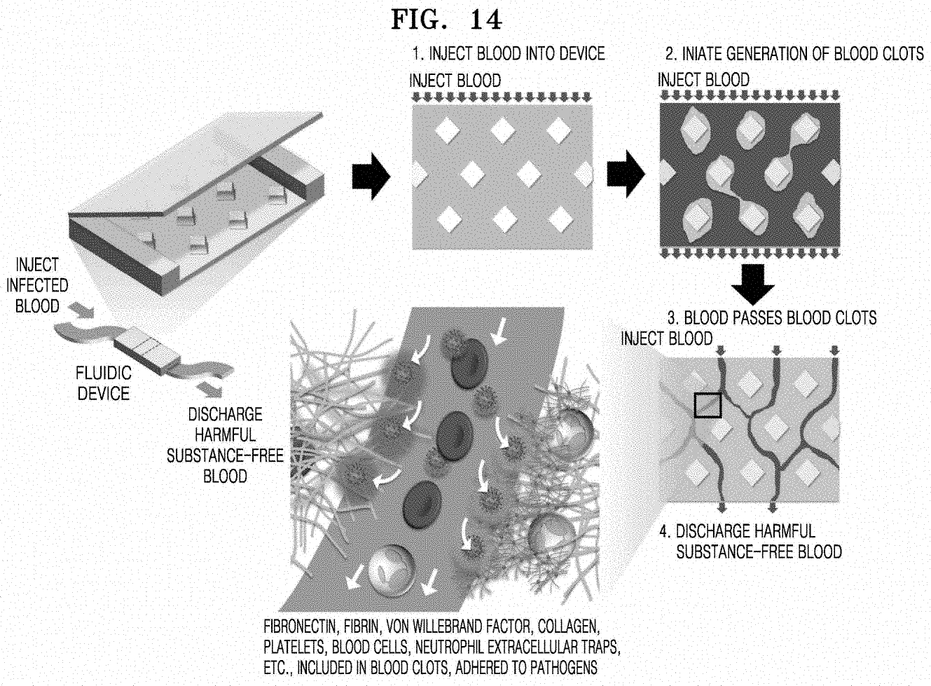

Referring to FIG. 14, when blood flows into the fluidic device 100, generation of blood clots starts as the blood flows into the blood-clot generating and fixing unit 140 configured to generate blood clots. Since a cross-sectional area of a fluidic channel of the blood-clot generating and fixing unit 140 is smaller than a cross-sectional area of a fluidic channel of the inlet 110, or the blood-clot generating and fixing unit 140 includes the microstructure 120 or particles, a surface shear rate of flowing blood increases, or blood clots are attached to the microstructure 120 or the particles, and thus blood clots may be generated from the flowing blood. The generated blood clots may be formed and immobilized on a surface or periphery of the microstructure 120, and the blood continues to flow to the surface or periphery of the microstructure 120, on which the blood clots are immobilized. At this time, blood cells, fibrinogen, fibronectin, von Willebrand factor, collagen, fibrin, platelets, or neutrophil extracellular traps (NETs) existing on surfaces of the blood clots or within the blood clots may capture (remove or bind to) harmful substances or blood clots in the blood.

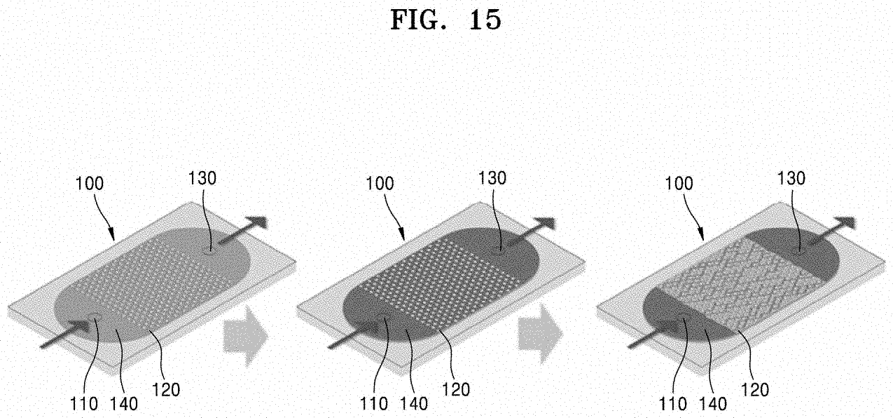

Referring to FIG. 15, when blood flows into the fluidic device 100, a flow rate of the blood changes as the flowing blood passes through a small cross-sectional area and low height of a fluidic channel of the blood-clot generating and fixing unit 140 of the fluidic device 100 or the plurality of microstructures 120, leading to an increase in shear rate of the blood. In addition, as the shear rate of the blood increases, blood clots may be more easily generated. Without being limited to a particular theory, a small cross-sectional area or low height of the blood-clot generating and fixing unit 140 of the fluidic device 100 and the plurality of microstructures 120 cause a change in flow rate of injected blood, and the greater the change, the higher the surface shear rate of the blood, resulting in generation of blood clots. The surface shear stress of the blood shows the largest value on a surface of a channel near the inlet 110 of the fluidic device 100 and also shows a large value between the plurality of microstructures 120, and thus blood clots may be immobilized on the surface or periphery of the microstructure 120.

### Experimental Example 1

### Analysis of Harmful Substance Removal Capability or Blood Clots Removal Capability by Inducing Shear Stress in Blood

Pathogen-containing blood was prepared to analyze whether harmful substances could be removed or blood clots could be fixed by inducing shear stress in flowing blood. First, random human blood (from Ulsan Blood Center of Korea Red Cross) was provided, and the blood was mixed with 10 mM calcium chloride, 1 U/ml of heparin sodium (Choongwae Pharma), and 36,800 CFU/ml of Staphylococcus aureus. 10 ml of the mixed solution was put into a 50 ml Eppendorf tube, followed by shaking (300 rpm, 15 sec interval, 4° C.) in a Thermomixer C (Eppendorf, USA), to prepare the pathogen-containing blood.

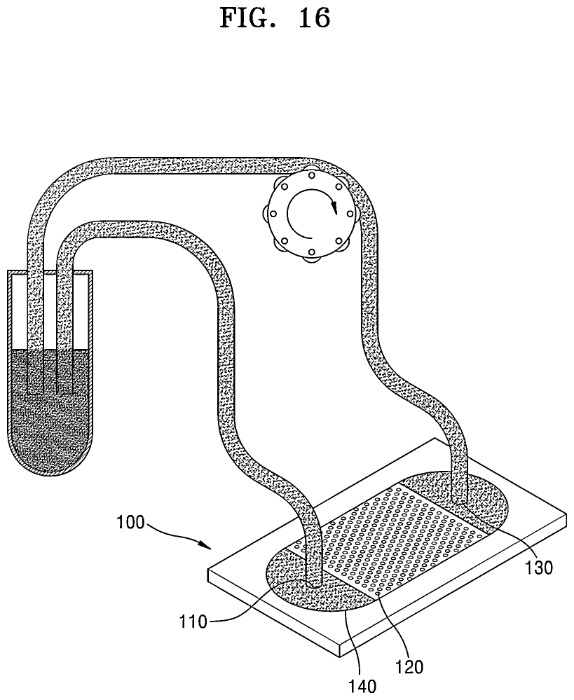

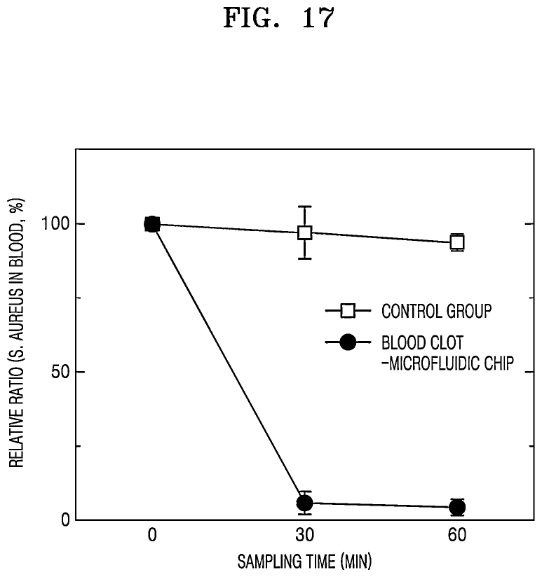

Subsequently, the prepared blood was circulated in a device capable of increasing shear stress of flowing blood. Specifically, as shown in FIG. 16, the prepared blood was circulated by connecting the fluidic device 100 (channel height=50 μm) according to an embodiment of the present disclosure of FIG. 2 with a silicone tube (Cole-Parmer, EW-95802-02) having an inner diameter of 1.52 mm and using a peristaltic pump (Reglo Digital). An injection speed of the blood was 20 ml/h. A sample of circulated blood was collected after 30 minutes of a pumping operation and after 60 minutes of a pumping operation, and a concentration of bacteria was measured by agar plating. As a control group, 10 ml of the prepared blood was put into the 50 ml tube, and after 30 minutes without any treatment and after 60 minutes without any treatment, a concentration of bacteria was measured by agar plating. Results thereof are shown in FIG. 17.

As shown in FIG. 17, in the case of the blood sample injected into the fluidic device 100 according to an embodiment of the present disclosure, the concentration of S. aureus was reduced by more than 95% after 30 minutes, compared to the initial concentration. In contrast, in the case of a blood sample from the control group, the concentration of S. aureus hardly decreased even after 60 minutes.

The above results indicate that harmful substances in blood may be non-specifically removed by generating blood clots by increasing shear stress of the blood.

### Experimental Example 2

### Results of Analysis of Fluid Simulations of Blood

In order to identify changes in flow and shear stress of blood in the fluidic device 100, fluid simulations in the fluidic devices 100 respectively having the microstructures of FIGS. 3 to 5 were analyzed.

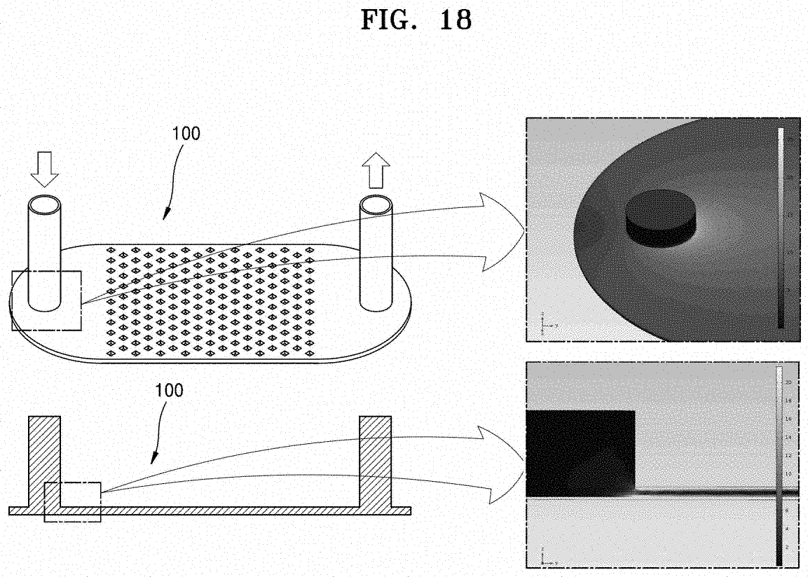

A surface shear rate and flow streamline were analyzed by analyzing the fluid simulations of the fluidic device 100 by using COMSOL Multiphysics 5.0, and results thereof and pictures of real experiments are shown in FIGS. 18 to 20.

As shown in FIG. 18, as flowing blood flowed from a tube having a large cross-sectional area into the fluidic device having a small cross-sectional area, a flow rate and shear stress increased due to a difference in cross-sectional area. Thus, substances involved in blood clotting, which include fibrin, von Willebrand factor, and platelets, were activated and generated blood clots. While passing through at least one microstructure provided in the fluidic device, the generated blood clots could adhere to a surface of a microstructure or could be trapped by minute gaps between microstructures, such that the blood clots could be fixed. Since other blood clots could adhere to surfaces of the fixed blood clots, blood clots may be gradually fixed more widely around the fixed blood clots.

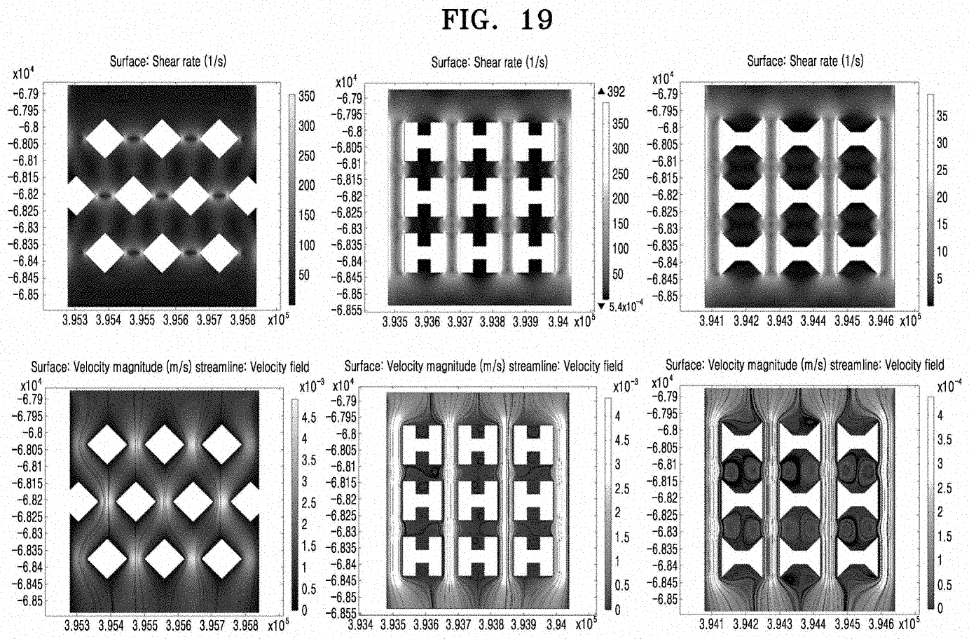

As shown in FIG. 19, the surface shear rate increased as the change in fluid velocity increased, and it was found that the higher the shear rate, the better the blood clots were generated, and the shear rate showed the largest value between microstructures. In addition, it was found that blood clots could be easily fixed in the case of the structure where the fluid velocity was low or the fluid was trapped. In addition, as a result of checking the flow streamline, it was found that the fluid was confined like a whirlpool in the downward and upward directions of the microstructure, and the generated blood clots could be efficiently immobilized between microstructures.

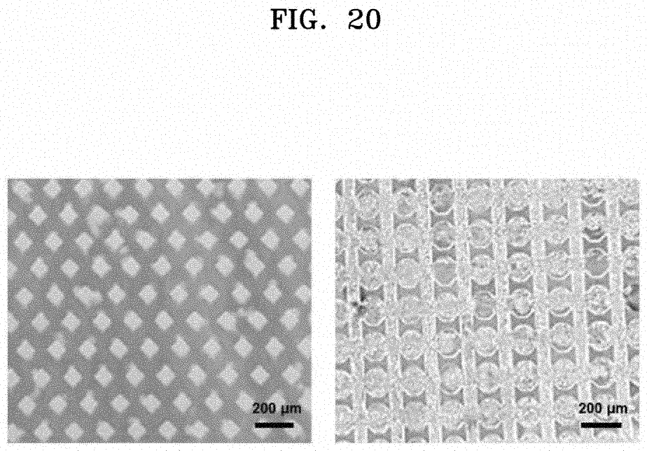

In addition, as shown in FIG. 20, as a result of checking generation of blood clots in two types of microstructures through actual experiments, in the case of the rhombic shape, it was found that blood clots were generated and fixed near the top and bottom of microstructures in the fluid direction, and in the case of the bow shape, it was found that blood clots were generated and fixed on the upper and lower sides of microstructures in the fluid direction.

### Experimental Example 3

### Analysis of Bacterial Removal Efficiency in Rat Blood

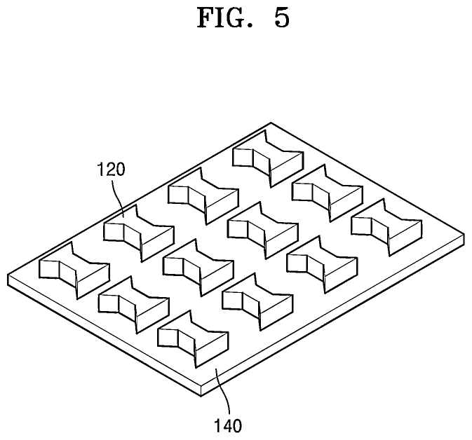

In the present embodiment, bacteria removal efficiency in rat blood by the fluidic device 100 having the structure of FIG. 5 was analyzed.

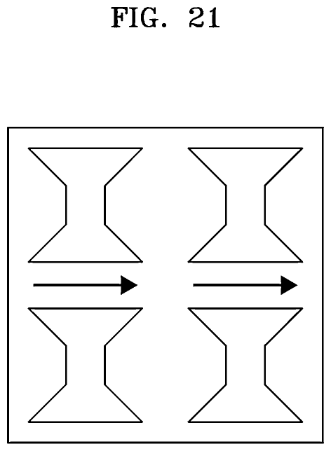

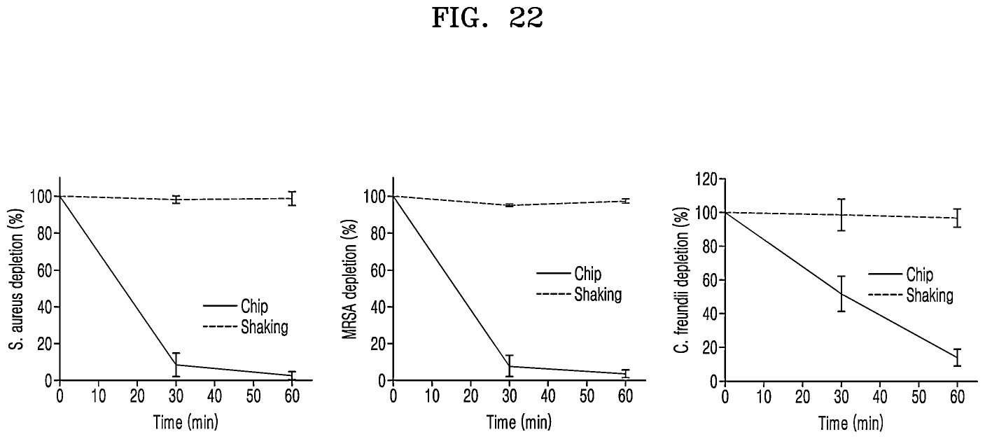

Specifically, a material of the fluidic device 100 was PDMS, and column structures (microstructures) having a shape as shown in FIG. 21 were arranged at intervals of 50 μm in a channel with approximately 5 cm wide, 2 cm long, and 50 μm high. Circular column structures having a diameter of 100 μm are arranged at intervals of 300 μm around the inlet and the outlet. To prepare a blood sample as an infection model, approximately 10 ml of blood was collected from a rat (Wistar, male, 8-10 weeks), and 1×10⁴ CFU/ml to 2×10⁴ CFU/ml of Staphylococcus aureus, 1×10⁴ CFU/ml to 2×10⁴CFU/ml of methicillin-resistant Staphylococcus aureus, and 1×10⁴ CFU/ml to 2×10⁴CFU/ml of Citrobacter freundii were each mixed with 10 ml of the blood. Similarly to FIG. 16, a Tygon tube (having an outer diameter of about 1.52 mm and an inner diameter of about 0.5 mm) was used to connect the inlet and outlet of the fluidic device together, and a peristaltic pump (Reglo Digital) was used to circulate and flow rat blood infected with each bacteria through a chip. At this time, flow rates were 20 ml/h for Staphylococcus aureus, 60 ml/h for methicillin-resistant Staphylococcus aureus, and 100 ml/h for Citrobacter freundii, respectively. The bacteria were mixed with the blood, a sample of the blood was collected before injection into the chip, after 30 minutes of circulation, and after 60 minutes of circulation, and concentrations of bacteria were measured by agar plating. As a control group, each bacteria was mixed with the same volume of 10 ml of the rat blood, followed by shaking in a Thermomixer C (Eppendorf, USA), for comparison with chip results, and results thereof are shown in FIG. 22.

FIG. 21 is a diagram of a shape of a microstructure of the blood-clot generating and fixing unit 140 of the fluidic device 100 according to an embodiment of the present disclosure, and arrows indicate a direction in which blood flows.

FIG. 22 shows graphs showing efficiency of removing various types of bacteria by the fluidic device 100 according to an embodiment of the present disclosure through decreases in relative quantity of the bacteria.

As shown in FIG. 22, compared to the initial concentration, the concentrations of Staphylococcus aureus and methicillin-resistant Staphylococcus aureus were reduced by at least 90% after 30 minutes, and the concentration of Citrobacter freundii was reduced by at least 90% after 60 minutes. In contrast, in the case of a blood sample from the control group, concentrations of bacteria hardly decreased even after 60 minutes.

### Experimental Example 4

### Hemolytic Reaction Reduction Effect by Removing Harmful Distances in Rat Blood

An experiment was conducted in the same manner as in Experimental Example 3, except that bacterial removal efficiency was evaluated by increasing a blood circulation time from 1 hour to 3 hours, and hemolysis was measured.

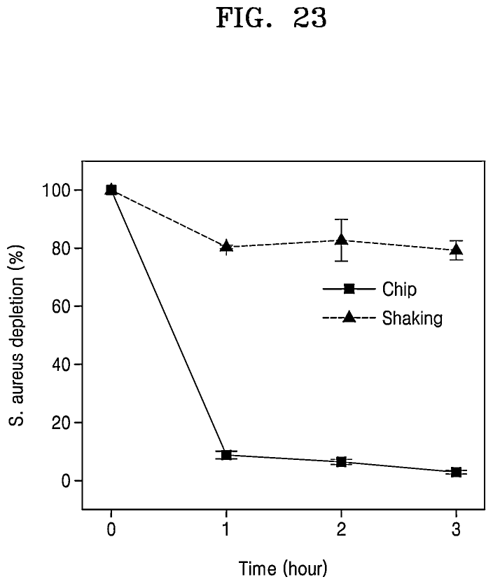

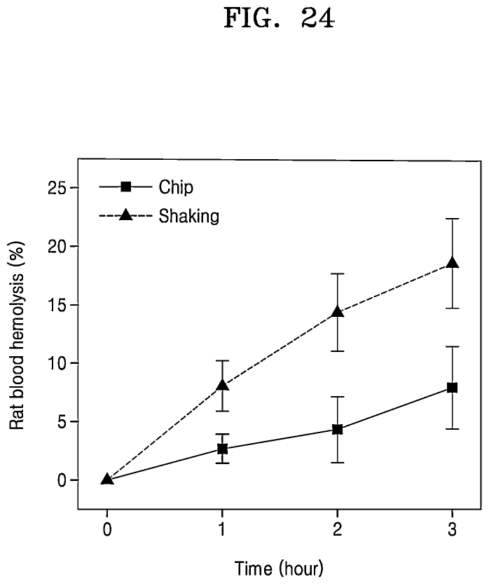

Specifically, a sample of the blood was collected before injection of the blood into the fluidic device 100, after 1 hour of circulation, after 2 hours of circulation, and after 3 hours of circulation, concentrations of the bacteria were measured by agar plating, and the degree of erythrocyte hemolysis was analyzed through absorbance measurement (Nanodrop One). Results thereof are shown in FIGS. 23 and 24.

FIG. 23 is a graph showing Staphylococcus aureus removal efficiency of the fluidic device 100 according to an embodiment of the present disclosure through a decrease in relative quantity of bacteria.

FIG. 24 is a graph of measuring hemolysis of rat blood by the fluidic device 100 according to an embodiment of the present disclosure.

As shown in FIGS. 23 and 24, compared to the initial concentration, Staphylococcus aureus was reduced by at least 90% after 1 hour. The control group in which the blood was shaken from the control group showed a small decrease. It was found that hemolysis occurred least in the chip conditions in which bacteria were most reduced. These results show that when bacteria-infected blood is flowed to the fluidic device 100, bacteria are removed and hemolysis of the blood is also reduced.

We claim:

1. A method of removing harmful substances in blood, which is performed by a fluidic device comprising:
an inlet into which isolated blood is injected; and
at least one blood-clot generating and fixing unit,
the method comprising:
injecting isolated blood into the inlet; and
removing harmful substances in blood by generating blood clots in the at least one blood-clot generating and fixing unit from the injected blood and fixing the blood clots;
wherein, in the removing of harmful substances in blood, the generated blood clots and the fixed blood clots capture the harmful substances in the blood being continuously injected, and
the removing of harmful substances in blood comprises allowing the blood to pass through the at least one blood-clot generating and fixing unit, injecting substances that generate blood clots into the blood, or contacting a surface on which the blood clots are generated with the blood.

2. The method of claim 1, wherein the generated blood clots and the fixed blood clots remove the blood clots in the blood by fixing the blood clots in the blood being continuously injected.

3. The method of claim 1, wherein the substances that generate blood clots comprise at least one selected from the group consisting of glass, polymers, metals, calcium, von Willebrand factor, blood cells, plasma, platelets, blood clotting factors, prothrombin, collagen, thrombin, fibronectin, fibrinogen, fibrin, neutrophil extracellular traps (NETs), antibiotics, heparin, heparan sulfate, chitosan, sialic acid, hyaluronic acid, polyethylene imine (PEI), dextran sulfate, chondroitin sulfate, dermatan sulfate, glycosaminoglycan, mannose, montmorillonite, bentonite, nanoclay, ligands including urea bonds, thiourea bonds, amide bonds, peptide bonds, amino groups, amide groups, carboxyl groups, pyridyl groups, pyrimidyl groups, and imidazole groups, polymyxin B, and polyamino compounds.

4. The method of claim 1, wherein the surface on which the blood clots are generated comprises at least one selected from the group consisting of glass, polymers, metals, calcium, von Willebrand factor, blood cells, plasma, platelets, blood clotting factors, prothrombin, collagen, thrombin, fibronectin, fibrinogen, fibrin, neutrophil extracellular traps (NETs), antibiotics, heparin, heparan sulfate, chitosan, sialic acid, hyaluronic acid, polyethylene imine (PEI), dextran sulfate, chondroitin sulfate, dermatan sulfate, glycosaminoglycan, mannose, montmorillonite, bentonite, nanoclay, ligands including urea bonds, thiourea bonds, amide bonds, peptide bonds, amino groups, amide groups, carboxyl groups, pyridyl groups, pyrimidyl groups, and imidazole groups, polymyxin B, and polyamino compounds.

5. The method of claim 1, wherein the at least one blood-clot generating and fixing unit generates the blood clots depending on shear stress of the blood according to a change in flow rate of the blood in which the flow rate of the blood increases or decreases as the blood passes through the at least one blood-clot generating and fixing unit.

6. The method of claim 1, wherein at least a portion of the at least one blood-clot generating and fixing unit comprises the surface on which the blood clots are generated, such that the generated blood clots are fixed to the at least one blood-clot generating and fixing unit.

7. The method of claim 6, wherein the surface on which the blood clots are generated comprises at least one selected from the group consisting of glass, polymers, metals, calcium, von Willebrand factor, blood cells, plasma, platelets, blood clotting factors, prothrombin, collagen, thrombin, fibronectin, fibrinogen, fibrin, neutrophil extracellular traps (NETs), antibiotics, heparin, heparan sulfate, chitosan, sialic acid, hyaluronic acid, polyethylene imine (PEI), dextran sulfate, chondroitin sulfate, dermatan sulfate, glycosaminoglycan, mannose, montmorillonite, bentonite, nanoclay, ligands including urea bonds, thiourea bonds, amide bonds, peptide bonds, amino groups, amide groups, carboxyl groups, pyridyl groups, pyrimidyl groups, and imidazole groups, polymyxin B, and polyamino compounds.

8. The method of claim 5, wherein the shear stress of the blood, capable of generating blood clots from the flowing blood is 1 dyne/cm² to 10,000 dyne/cm².

9. The method of claim 1, wherein the blood clots comprise at least one selected from the group consisting of blood cells, plasma, platelets, calcium, von Willebrand factor, blood clotting factors, prothrombin, collagen, thrombin, fibronectin, fibrinogen, fibrin, neutrophil extracellular traps (NETs), antibiotics, heparin, heparan sulfate, chitosan, sialic acid, hyaluronic acid, polyethylene imine (PEI), dextran sulfate, chondroitin sulfate, dermatan sulfate, glycosaminoglycan, mannose, montmorillonite, bentonite, nanoclay, ligands including urea bonds, thiourea bonds, amide bonds, peptide bonds, amino groups, amide groups, carboxyl groups, pyridyl groups, pyrimidyl groups, and imidazole groups, polymyxin B, and polyamino compounds.

10. The method of claim 1, wherein the harmful substances comprise at least one selected from the group consisting of infectious substances, cancers, tumors, cancer cells, cancer-inducing substances, cancer-related factors, and extracellular vesicles (exosomes).

11. The method of claim 1, wherein the fluidic device further comprises an outlet to discharge the blood from the at least one blood-clot generating and fixing unit.

12. The method of claim 1, wherein the at least one blood-clot generating and fixing unit comprises microstructures protruding from an upper surface of the substrate, hollow fiber, or particles that are immobilized or unfixed in the at least one blood-clot generating and fixing unit and capable of inducing shear stress of the blood.

13. The method of claim 12, wherein a cross section of the microstructures is n-gonal or amorphous, and n is 3 to 12.

14. The method of claim 12, wherein a height or interval of the microstructures is 10 μm to 10,000 μm.

15. The method of claim 12, wherein the fluidic device further comprises:
a fluidic channel arranged to correspond to the at least one blood-clot generating and fixing unit and through which the blood moves, and
a height of the microstructures is equal to or less than a height of the fluidic channel.

16. The method of claim 12, wherein the particles are rod-shaped, bead-shaped, or fibers.

17. The method of claim 1, wherein the fluidic device further comprises:
fluidic channels arranged to respectively correspond to the inlet and the at least one blood-clot generating and fixing unit and through which the blood moves, and
a cross-sectional area of a fluidic channel of the at least one blood-clot generating and fixing unit is smaller than a cross-sectional area of a fluidic channel of the inlet, or a height of the fluidic channel of the at least one blood-clot generating and fixing unit is smaller than a diameter of a cross section of the fluidic channel of the inlet,
wherein as the blood flows from the inlet to the at least one blood-clot generating and fixing unit, stress shear of the blood is induced and blood clots are generated.

18. The method of claim 17, wherein the at least one blood-clot generating and fixing unit comprises a structure in which the cross-sectional area of the fluidic channel is changed at least once.

19. The method of claim 1, wherein the at least one blood-clot generating and fixing unit comprises a plurality of layers or a plurality of tubes.

20. The method of claim 17, wherein the fluidic device further comprises a blood clot filter capable of fixing blood clots to the at least one blood-clot generating and fixing unit or an outlet.
